(19) United States
(12) Patent Application Publication
(10) Pub. No.: US 2016/0033463 A1
(43) Pub. Date: Feb. 4, 2016

(54) METHOD AND APPARATUS FOR A COOKING OIL QUALITY SENSOR

(71) Applicant: FRYMASTER L.L.C., Shreveport, LA (US)

(72) Inventors: Ezban F. ROBERTSON, Shreveport, LA (US); Eric N. PITCHFORD, Shreveport, LA (US); John CALOW, Bossier City, LA (US)

(21) Appl. No.: 14/815,008

(22) Filed: Jul. 31, 2015

Related U.S. Application Data
(60) Provisional application No. 62/032,251, filed on Aug. 1, 2014.

(51) Int. Cl. G01N 33/03; A23L 1/015; A23L 1/01

(52) U.S. Cl. CPC G01N 33/03; A23L 1/0107; A23L 1/015; A23V 2002/00

(57) ABSTRACT

A fryer includes a fryer pot, a filter pan connected to the fryer pot by a drain conduit and a return conduit forming a filtration loop, a cooking oil quality sensor being in the filtration loop, and a controller that controls operation of a filtration cycle of the fryer. The filtration cycle has a circulation sequence and a fill sequence. The circulation sequence circulates cooking oil through the filtration loop and the fill sequence fills the fryer pot with the cooking oil from the filter pan. The controller stops the fill sequence after filling the fryer pot with a predetermined amount of cooking oil during a partial fill and resumes the fill sequence after a predetermined amount time elapses to complete the fill sequence. The cooking oil quality sensor measures a cooking oil quality to obtain a cooking oil quality measurement during the predetermined amount time.

This application claims the benefit of U.S. Provisional Application No. 62/032,251, filed Aug. 1, 2014. The contents of U.S. Provisional Application No. 62/032,251, filed Aug. 1, 2014, are hereby incorporated by reference herein in their entirety.

### BACKGROUND OF THE DISCLOSURE

1. Field of the Disclosure

This disclosure relates to a cooking oil quality sensor that is installed in a fryer for the purpose of indicating when the cooking oil should be changed for one or more fryer pots. This disclosure, more particularly, relates to a cooking oil quality sensor that measures an electrical property of the cooking oil and is disposed in a filtration loop of a fryer that is external to the one or more fryer pots.

2. Description of Related Art

During use, the cooking oil in a fryer is degraded and loses its proper cooking capacity. Specifically, the degradation is caused by oxidation, cyclic temperature increases and hydrolysis from released water. Impurities that are generated during the frying process are collectively called total polar materials (TPMs) or total polar compounds (TPCs). The TPMs are created during the deep-frying process as triglycerides break into free fatty acids and lipid molecule residues. These substances are characterized by an increased polarity and dielectric constant compared to the original triglycerides in the cooking oil. Thus, an increased capacitance measurement of the cooking oil is indicative of an increased level of TPMs in the cooking oil.

There are several methods for testing the quality of cooking oil. Simple methods such as testing the taste, smell and color of the cooking oil are excessively subjective, inaccurate and too time consuming. Other methods test the smoke point or viscosity of the cooking oil. Again, while these measurements are fairly simple, they are too dependent on factors such as cooking oil type and cooking oil debris to be universally reliable.

Processes that include chemical or chromatographic methods are generally more comprehensive and accurate than the simpler methods. For example, currently the most widely used test tests the fatty acids that are released from glycerines during the frying process. This test depends strongly on the moisture of the frying goods. Testing for polymeric triglycerides that are formed from frying triglycerides is often time consuming and expensive.

Accordingly, there is a need for detection of the level of all deterioration products or TPMs in a fryer.

### SUMMARY

A fryer is provided that includes a fryer pot, a filter pan connected to the fryer pot by a drain conduit and a return conduit forming a filtration loop, a cooking oil quality sensor being in the filtration loop, and a controller that controls operation of a filtration cycle of the fryer. The filtration cycle has a circulation sequence and a fill sequence. The circulation sequence circulates cooking oil through the filtration loop and the fill sequence fills the fryer pot with the cooking oil from the filter pan. The controller stops the fill sequence after filling the fryer pot with a predetermined amount of cooking oil during a partial fill and resumes the fill sequence after a predetermined amount time elapses to complete the fill sequence. The cooking oil quality sensor measures a cooking oil quality to obtain a cooking oil quality measurement during the predetermined amount time.

A fryer is also provided that includes a fryer pot, a filter pan connected to the fryer pot by a drain conduit and a return conduit forming a filtration loop, a cooking oil quality sensor being in the filtration loop, and a controller that controls operation of a filtration cycle of the fryer. The controller has data stored on a memory of a plurality of different types of cooking oil. The controller communicates the data to the cooking oil quality sensor based on one of the different types of cooking oil present in the filtration loop.

A method of measuring cooking oil quality in a fryer is also provided that includes commencing a filter cycle in a fryer; starting a fill sequence to fill a fryer pot with cooking oil; stopping the fill sequence for a predetermined amount of time; acquiring total polar materials data during the predetermined amount of time; and resuming the fill sequence.

The above-described and other advantages and features of the present disclosure will be appreciated and understood by those skilled in the art from the following detailed description, drawings, and appended claims.

### BRIEF DESCRIPTION OF THE DRAWINGS

FIG. 1 illustrates an exemplary fryer housing a sensor of the present disclosure.

FIG. 2 illustrates a cooking oil quality sensor according to the present disclosure incorporated into a return pipe of a filtration loop of the fryer of FIG. 1.

FIG. 3 illustrates the cooking oil quality sensor according to the present disclosure incorporated into a drain pipe of the filtration loop of the fryer of FIG. 1.

FIG. 4 illustrates the cooking oil quality sensor according to the present disclosure incorporated into a filter pan of the filtration loop of the fryer of FIG. 1.

FIG. 5 is a block diagram of the filtration loop having a dispose valve between the filter pan and the cooking oil quality sensor.

FIG. 6 is a block diagram of the filtration loop having a dispose valve at the filter pan.

FIG. 7 is a block diagram of the filtration loop having a dispose valve between the cooking oil quality sensor and a fryer pot.

FIG. 8 is a schematic diagram of a controller of a fryer system and the cooking oil quality sensor.

FIG. 9 is a sequence diagram of data communicated between the controller of the fryer system and the cooking oil quality sensor to measure a total polar materials (“TPM”) sample.

FIG. 10 is a sequence diagram of data communicated between the controller of the fryer system and the cooking oil quality sensor to acquire a TPM sample.

FIG. 11 is a flow diagram of a method of measuring cooking oil quality of the present disclosure.

FIG. 12 is a flow diagram of a method of determining a cooking oil formula of the present disclosure.

FIG. 13 is an illustration of a computer system used to implement the disclosed embodiment.

### DETAILED DESCRIPTION OF THE DISCLOSURE

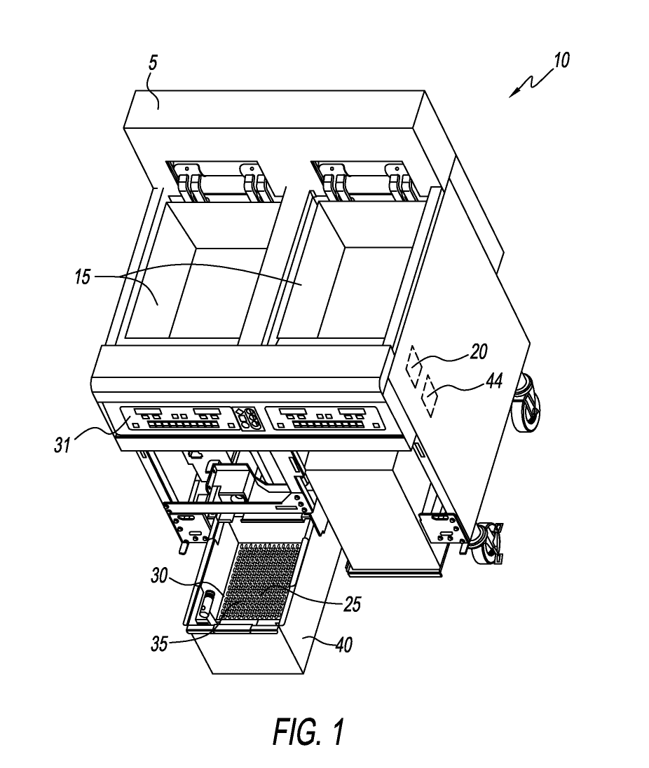

Referring to FIG. 1, an illustration of an exemplary fryer is shown, and generally represented by reference numeral 10. Deep fryer 10 has a housing 5, a pair of fryer pots 15 and a pair of filter pans 40. Each of the pair of filter pans 40 contains a pre-filtering medium, such as a sieve 35 that is used to remove large particles from the used cooking oil. Alternatively, both fryer pots 15 could share a common filter and return system. While fryer 10 is shown as only having two fryer pots 15, there could be as many as twelve fryer pots depending upon the needs of the food service professional. Fryer 10 also has a controller 20 for monitoring and maintaining overall operation the fryer 10. Deep fryer housing 5, also has a display panel 31 that displays various measurements of deep fryer and accepts input for programming of controller 20. The present application is not limited to cooking oil, thus fat or shortening could also be used in the present application.

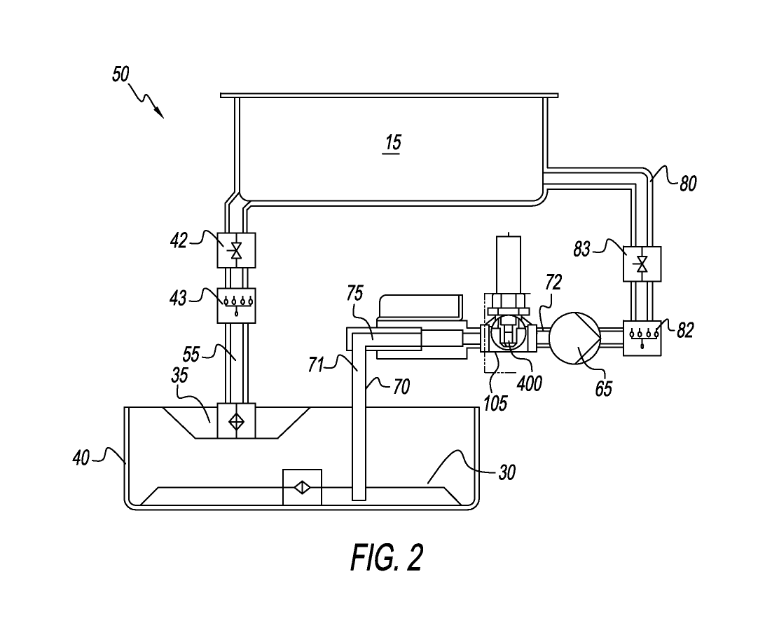

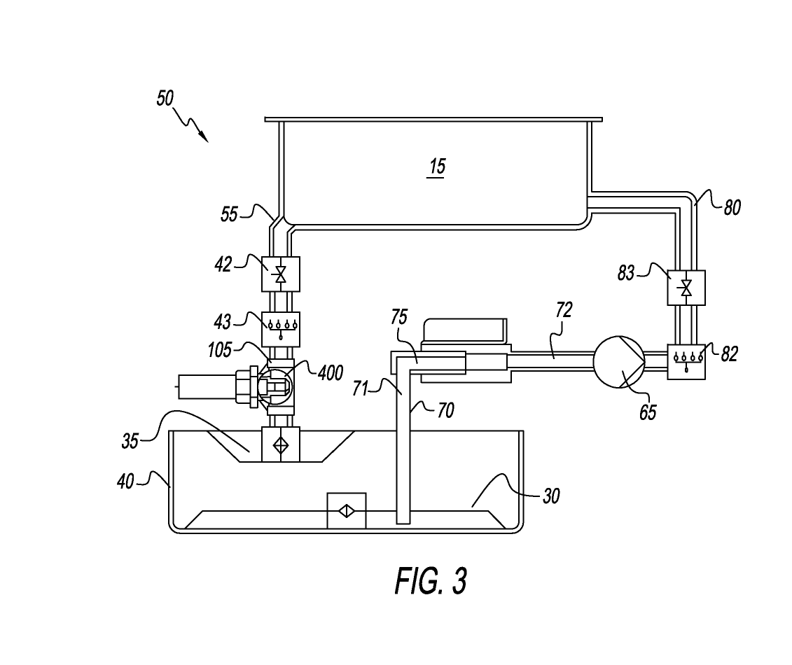

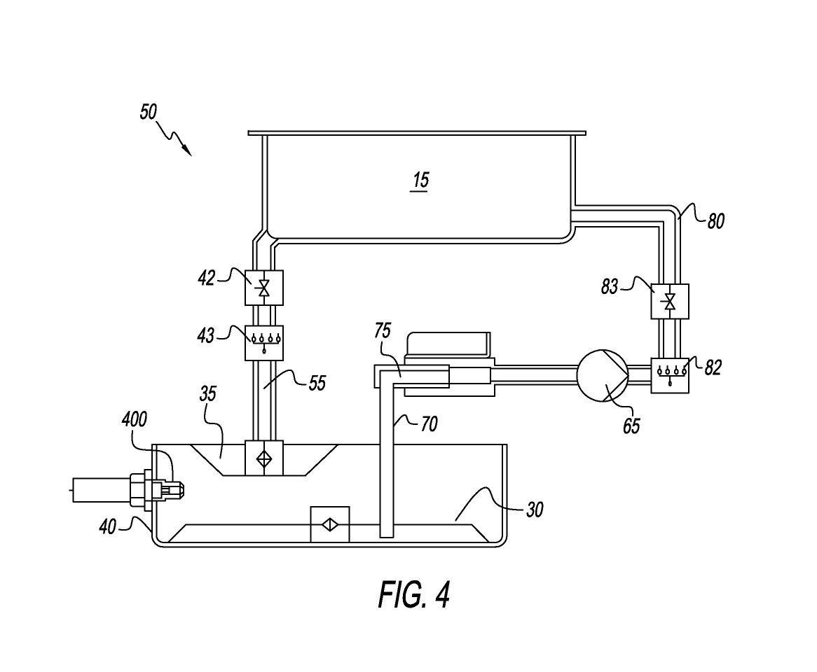

Referring to FIG. 2, filtration loop 50 of fryer 10 incorporates a sensor and is shown, and referenced using reference numeral 400. Sensor 400 is shown in the return line 70 of filtration loop 50; however, sensor 400 preferably is disposed along filtration loop 50 external to fryer 10, in accordance with the present disclosure. Thus, sensor 400 is disposed in filtration loop 50 external to fryer pot 15 independent of the configuration of filtration loop 50, as shown in FIGS. 3 through 4. Further, sensor 400 is capable of measuring an electrical property of cooking oil 75 such, as the dielectric constant, of cooking oil. Sensor 400 is, for example, one of a capacitance sensor, an open ended coaxial sensor, a conductivity or a resonant type sensor.

Referring again to FIG. 2, filtration loop 50 has a drain line 55, and a pre-filtration sieve 35, and a fine filtration pad 30. Cooking oil 75 is returned through plumbing 70 by pump 65. Prior to reaching pump 65, sensor 400 in flow of returning filtered cooking oil 75 is able to sample the an electrical property as cooking oil 75 is being returned to fryer pot 15. A filtration loop that services multiple fryer pots would have a gate valve 42 and common drain plumbing 43 disposed upstream of pan 40 to collect used cooking oil 75 from multiple fryer pots 15. Similarly, a return line splitter 82 and a valve 83 would direct filtered cooking oil to specific fryer pots 15.

Referring to FIG. 3, sensor 400 is disposed in drain pipe 55 of filtration loop 50. In this embodiment, an electrical property of cooking oil 75 is sampled as cooking oil 75 is drained from fryer pot 15. A filtration loop 50 that services multiple fryer pots 15 would have a gate valve 42 and common drain plumbing 43 disposed upstream of pan 40 to collect used cooking oil 75 from multiple fryer pots. Similarly, a return line splitter 82 and a valve 83 would direct filtered cooking oil to specific fryer pots 15.

Referring to FIGS. 2 and 3, sensor 400 is contained within adapter 105 that extends within housing 5 generally beneath fryer pot 15. Adapter 105 is connected in return-line of cooking oil of pipe 70. Adapter 105 is preferably connected between two portions of return pipe 70, upstream portion 71 and downstream portion 72, in a mating relationship via mating threads disposed on interconnecting portions thereof. Cooking oil sensor 400 extends within adapter 105 and is positioned to lie in the stream of flow of cooking oil 75, such that the flow of cooking oil 75 from upstream portion 71, through adapter 105 to downstream portion 72 is uninterrupted. Additionally, the flow of cooking oil 75 is coincident with longitudinal axis of upstream portion 70, downstream portion 72 and adapter 105 installed between portions 71 and 72. Cooking oil sensor 400 extends within and is protected by adapter 105.

Referring to FIG. 4, sensor 400 is disposed in filtration loop 50, in filter pan 40. In this configuration, the dielectric constant of filtered cooking oil is sampled in pan 40 prior to passing through filtration pad 30 and returning to fryer pot 15.

Cooking oil sensor 400 is located in an adapter 105 in the filtration loop of fryer pot 15 as shown in FIGS. 2 and 3. Sensor 400 is located to measure sample an electrical property of cooking oil 75 before it re-enters fryer pot 15, independent of its location external to fryer pot 15. When the triglycerides of filtered cooking oil 75 break into fatty acids and lipid molecules during the heating and cooking cycles the polarity of cooking oil 75 increases. The accumulation of polar materials lowers the insulating properties of cooking oil and elevates the dielectric constant of cooking oil 75 to higher values. This increased polarity correlates with an increased dielectric constant of cooking oil 75. Thus, sensor 400 is able to measure the change of the TPM values by measuring the dielectric constant of cooking oil 75 as pump 65 returns cooking oil to fryer pot 15. When sensor 400 detects an unacceptable level of TPMs an indication is provided to an operator to change the cooking oil. Thus, sensor 400 ensures that cooking oil 75 is not wasted by being prematurely changed or overused thereby tainting food and harming consumers.

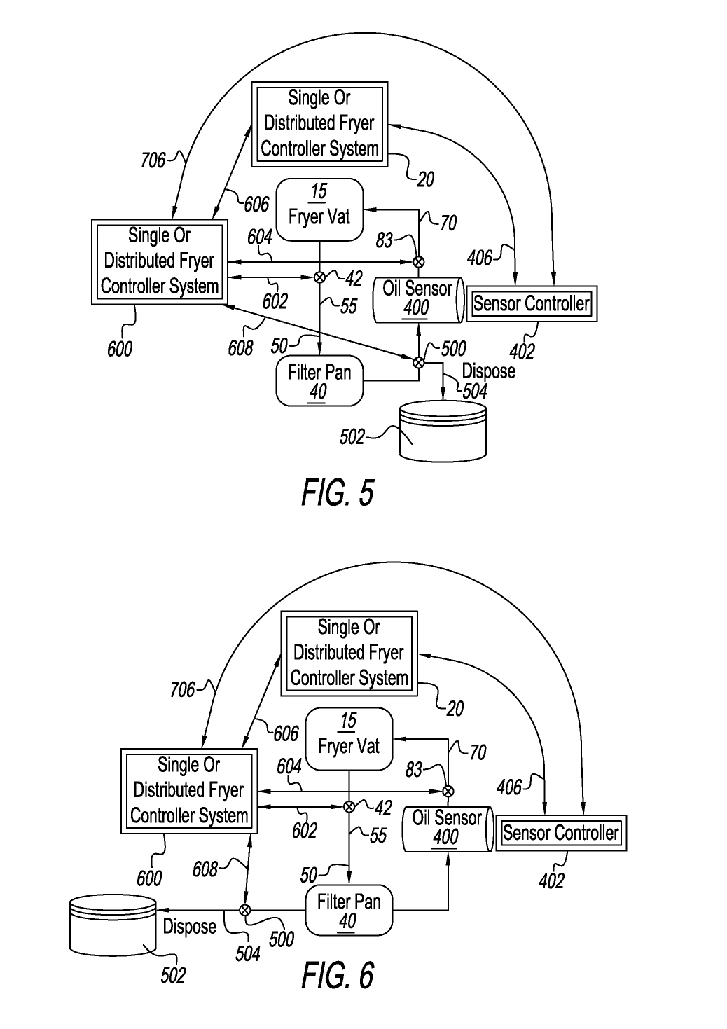

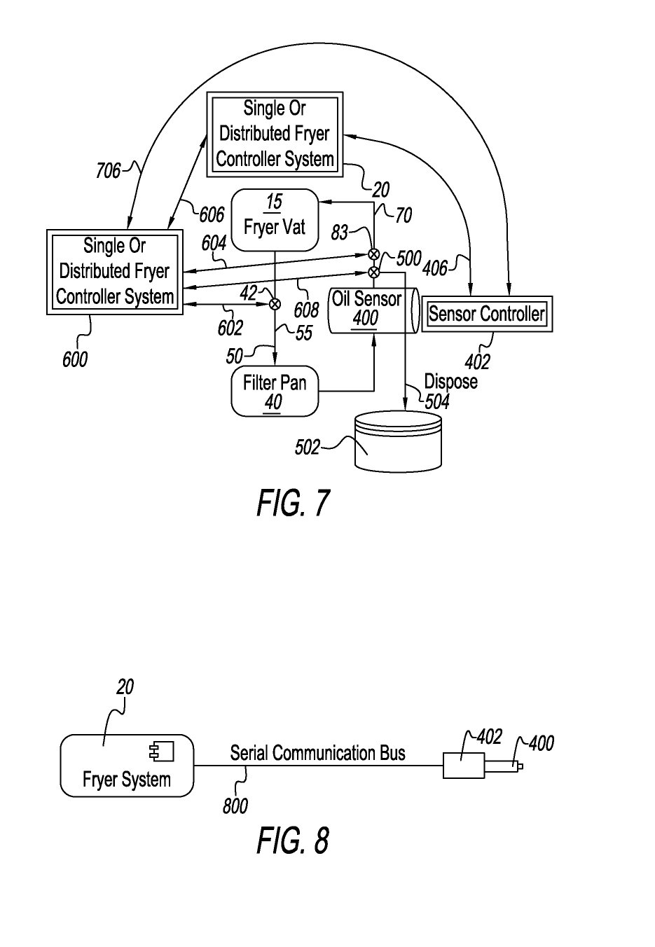

Referring to FIGS. 5-7, sensor 400 has a sensor controller 402 and fryer 10 has a disposal valve 500 that connects filtration loop 50 to a disposal container 502. Disposal valve 500 is in return line 70 between filter pan 40 and sensor 400 in FIG. 5. As shown in FIG. 6, disposal valve 500 can be in filter pan 40. As shown in FIG. 7, disposal valve 500 can be in return line 70 between valve 83 and sensor 400. When disposal valve 500 is in an opened position, cooking oil is drained from filtration loop 50 to disposal container 502 through a disposal conduit 504 rather than being circulated to fill fryer pot 15. When valve 500 is in a closed position, the cooking oil is maintained in filtration loop 50.

Fryer 10 has a valve controller 600. Valve controller 600 actuates gate valve 42, valve 83 and disposal valve 500. Valve controller 600 actuates gate valve 42, valve 83 and disposal valve 500 to move each of gate valve 42, valve 83 and disposal valve 500 between an open position in which cooking oil can pass through each gate valve 42, valve 83 and disposal valve 500 and a closed position in which cooking oil is prevented from passing through each of gate valve 42, valve 83 and disposal valve 500. Each of gate valve 42, valve 83 and disposal valve 500 is actuated independently from one another. Valve controller 600 communicates output to gate valve 42 and valve controller 600 receives input from gate valve 42, as shown by arrow 602, to actuate gate valve 42. Valve controller 600 communicates output to valve 83 and valve controller 600 receives input from valve 83, as shown by arrow 604, to actuate valve 83. Valve controller 600 communicates output to disposal valve 500 and valve controller 600 receives input from disposal valve 500, as shown by arrow 608, to actuate disposal valve 500. Valve controller 600 may be a single controller. Alternatively, each of gate valve 42, valve 83 and disposal valve 500 may have a separate controller.

Controller 20 communicates output to sensor controller 402 and receives output communicated from sensor controller 402, as shown by arrow 406. Controller 20 communicates output to valve controller 600 and receives output communicated from valve controller 600, as shown by arrow 606. Sensor controller 402 communicates output to valve controller 600 and receives output communicated from valve controller 600, as shown by arrow 706.

Referring to FIG. 8, sensor 400 is interfaced to controller 20 in a serial communication channel. Controller 20 is connected to sensor 400 by a serial communication bus 800. Controller 20 communicates output to sensor controller 402 and receives output communicated from sensor controller 402 through serial communication bus 800. Through this serial link, commands and system data are sent to and from controller 20 and sensor 400 to acquire TPM measurements as well as starting and stopping measurement activities. This serial link is also used to transfer sensor firmware as well as updating the selection of cooking oil curve formulae from controller 20 to sensor 400. The arbitration subsystem of controller 20 arbitrates the use of the filtration subsystem of controller 20 and subsequently sensor 400 for activating or deactivating TPM acquisitions. The arbitration subsystem detects whether a filter cycle may be commenced. The TPM values from sensor 400 are also provided to controller 20 through this serial interface.

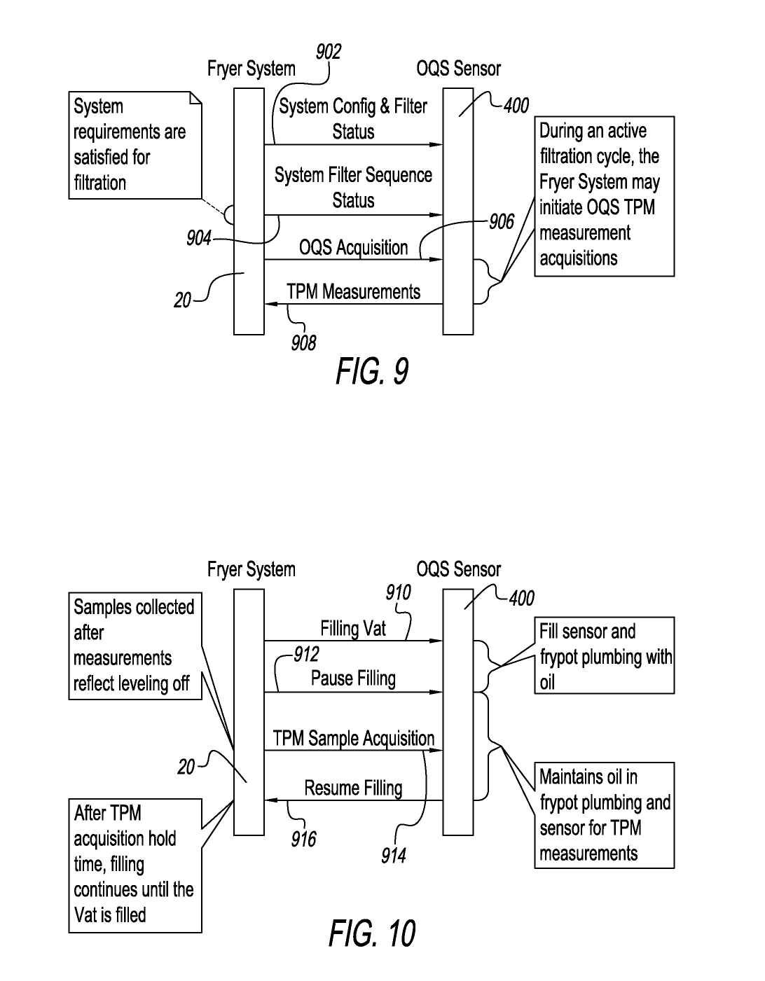

Referring to FIG. 9, when controller 20 determines predetermined system requirements have been met to commence a filtration cycle, controller 20 communicates to sensor 400 a system configuration and filter status, as shown by arrow 902, and a system filter sequence status, as shown by arrow 904. For example, controller 20 can communicate to sensor 400 information such as that fryer 10 currently is in a filter cycle, that the filter cycle is active, and/or that the filter cycle is at a beginning or end of the filter cycle in communications 902 and 904. During the filtration cycle, controller 20 can send a command to sensor 400 to acquire TPM measurements, as shown by arrow 906. Upon receipt of the command to acquire a TPM measurement, sensor 400 measures TPM in cooking oil contacting sensor 400. Sensor 400 communicates a TPM measurement to controller 20, as shown by arrow 908.

Referring to FIG. 10, controller 20 communicates to sensor 400 that fryer pot 15 is being filled with cooking oil during a fill sequence of a filtration cycle, as shown by arrow 910. The filtration cycle has a circulation sequence, and, after the circulation sequence is completed, a fill sequence is carried out. Controller 20 pauses or stops the fill sequence prior to completion of filling of fryer pot 15 with the cooking oil. When the fill sequence is stopped, controller 20 communicates to sensor 400 that the fill sequence is stopped and a command to acquire a TPM measurement, as shown by arrow 912. Upon receipt of the command acquire the TPM measurement, sensor 400 measures TPM in cooking oil contacting sensor 400. After sensor 400 measures the TPM in the cooking oil, sensor 400 communicates the TPM measurement to controller 20, as shown by arrow 914. Controller 20 communicates to sensor 400 that the fill sequence has resumed to complete fill of fry pot 15 with the cooking oil.

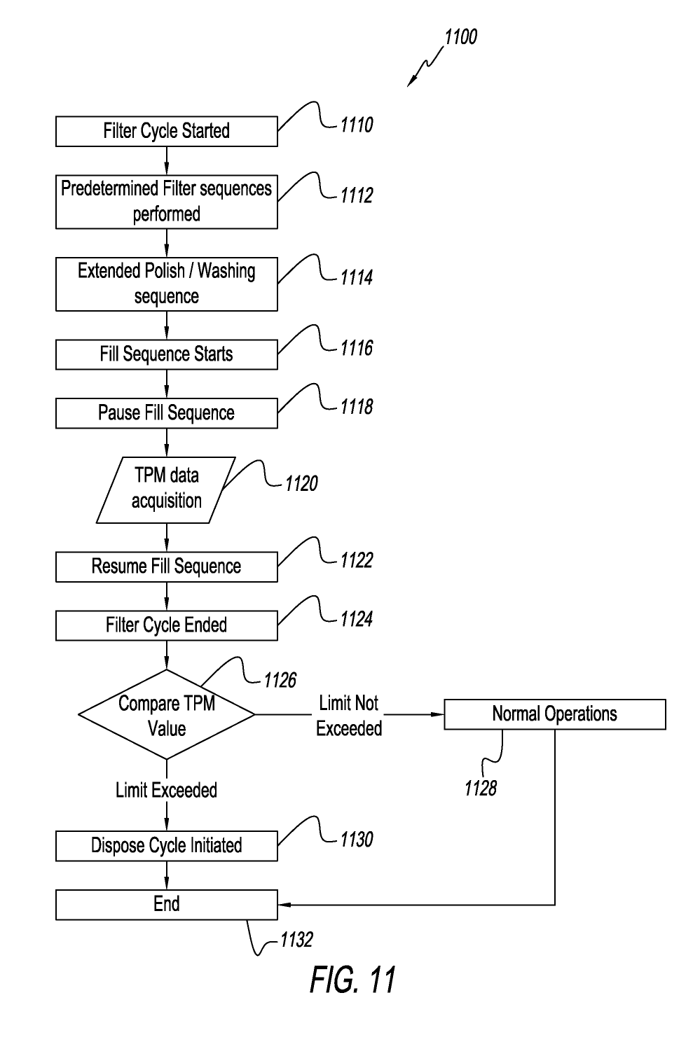

Referring to FIG. 11, a method 1100 of measuring cooking oil quality in fryer 10 is shown. A filter cycle is started in step 1110. Method 1100 proceeds from step 1110 to step 1112. Predetermined filter sequences are performed in step 1112. Method 1100 proceeds from step 1112 to step 1114. Extended polish or washing sequence is performed during step 1114. Method 1100 proceeds from step 1114 to step 1116. A fill sequence is commenced during step 1116 in which fryer pot 15 is filled with the cooking oil. Method 1100 proceeds from step 1116 to step 1118. The fill sequence is paused in step 1118 so that cooking oil does not fill fryer pot 15 and fryer pot 15 remains partially filled. Method 1100 proceeds from step 1118 to step 1120. TPM data is acquired in step 1120. Method 1100 proceeds from step 1120 to step 1122. The fill sequence is resumed in step 1122 so that cooking oil fills fryer pot 15. Method 1100 proceeds from step 1122 to step 1124. The filter cycle is ended in step 1124. Method 1100 proceeds from step 1124 to step 1126. The TPM data acquired in step 1120 is compared to a predetermined TPM value in step 1126. If the TPM data acquired in step 1120 does not exceed a predetermined limit or the predetermined TPM value of step 1126, method 1100 proceeds from step 1126 to step 1128. Fryer 10 proceeds with normal operations in step 1128. Method 1100 proceeds from step 1128 to step 1132 and method 1100 is ended. If the TPM data acquired in step 1120 does exceed the predetermined limit or the predetermined TPM value of step 1126, method 1100 proceeds from step 1126 to step 1130. In step 1130, a dispose cycle is initiated to dispose of at least a portion of the cooking oil out of fryer 10. Alternatively, a top off cycle can be initiated to add new cooking oil to the exiting cooking oil in fryer pot 15 instead of commencing a dispose cycle. Method 1100 proceeds from step 1130 to step 1132 and method 1100 is ended.

Proper integration of sensor 400 into fryer 10 is vital for reliable cooking oil quality measurements. There are varieties of cooking oils and each has a different cooking oil formula which exhibits unique dielectric properties. Hence, the TPM quality of the cooking oil is also dependent on the cooking oil formula. Sensor 400 measuring the TPM levels of the cooking oil must take each formula into consideration. Sensor 400 is integrated into a single or battery of fryers as sensor 400 interacts with other subsystems. Sensor 400 is communicated a cooking oil type that is selected by a user. Sensor 400 is communicated an update for various cooking oil types. For example, controller 20 sends sensor 400 cooking oil type information.

Sensor 400 is strategically placed in a common inline path of cooking oil return to any fryer pots 15 in fryer 10. To acquire a reliable measurement from sensor 400, a sequence of events that involves system handshaking with sensor 400 must occur. This handshake protocol involves a fryer user interface, a filtration subsystem of controller 20 and the arbitration subsystem of controller 20.

When a controller 20 is equipped with sensor 400, fryer 10 is enabled for TPM measurements with options available through various filtration sequences. TPM measurements are performed during any of the filter sequences once the particular filter cycle has been initiated. The initiation of the filter cycle can occur manually through the user interface or via a previously programmed event. This preprogrammed event can either be time or fryer statistics driven of fryer 10. There are several sequences to a filtration cycle. Several filtration cycles are often referred to as types of filter or filtration cycles. Of the filtration sequences, two basic sequences are involved in the cooking oil quality measurement; a cooking oil circulation sequence—sometimes referred to as filter, polish or washing sequences—, and a filling sequence. The circulation sequence allows the cooking oil at a predetermined temperature(s) to circulate through fryer pot 15 being filtered for several minutes depending on the filter type. This allows a temperature measurement of TPM to attain a desired and constant level. At the end of this filtering sequence, the filling sequence commences, during which cooking oil quality measurements are taken by sensor 400.

The filling sequence of fryer 10 occurs in two phases. The first phase of the cycle performs a partial fill of fry pot 15 and has duration of several seconds time, for example, between 0 seconds to 120 seconds. This clears air from the filter channels to the filtering frypot, as well as maintaining a volume of cooking oil in fry pot 15, sensor 400 and filter pan 40. This partial fill of fry pot 15 is maintained additionally for several seconds, for example, between 0 seconds to 120 seconds. During this time, TPM measurement samples are taken by sensor 400 from which a final TPM value results. The final TPM value is a result of an average of more than one sample taken over a specific time-frame between 0 seconds to 120 seconds, and, further, for example, between 1 and 100 samples. The final TPM value is shown on the user interface when acquisition is completed. TPM values are stored and kept for several days. At the end of the partial fill, TPM acquisition sequence, a second phase of the fill sequence commences which continues to fill the fry pot 15 until the fill requirements are satisfied.

Sensor 400 can repeatedly sample TPM in cooking filtered cooking oil 75, these data are sent to controller 20. The measurements are averaged over the duration. Thus, the calculated averaged value of the TPMs can be calculated and compared to known accurate values to detect the dielectric constant of the cooking oil. Controller 20 is capable of storing acceptable dielectric values of clean cooking oil for comparison to the measured values. Should the dielectric constant of filtered cooking oil 75 exceed a predetermined threshold, an indicator, such as an audible or visible alarm, is engaged. Additionally, display on display panel 31 shows measurements.

When a filtration cycle is initiated by controller 20, the filtration subsystem of controller 20 is notified of a type of filtration that was initiated. Sensor 400 is also notified by controller 20 when a filtration cycle is initiated which allows sensor 400 to be staged and ready for TPM acquisition. Other configuration parameters, for example, whether the filter cycle is active, whether the filter cycle is at a beginning of the filter cycle, or whether the filter cycle is at an end of the filter cycle, are also provided to the filtration subsystem assuring a correct sequence is initiated as selected. These configuration parameters, depending on the filtration type, allow the user to bypass the cooking oil quality measurement by sensor 400 should they choose to do so.

At the end of circulation sequence, gate valve 42 is closed to allow the filling cycle to begin. At the end of a first part of the fill sequence (the partial fill sequence), the filtration subsystem disables pump 65 and allows the cooking oil to remain stagnant in sensor 400 for a period of time, for example, 0 s-120 s. The TPM acquisition by sensor 400 commences over a period of time with specific delays allowing TPM measurement levels to stabilize and produce values with greater accuracy.

Dependent on the customer and/or region, when a TPM measurement acquired by sensor 400 exceeds a predetermined limit, controller 20 initiates a cooking oil dispose cycle to discard the cooking oil in filtration loop 50 that is poor quality cooking oil. The user however, is provided the option to cancel this dispose cycle or continue disposing the cooking oil, for example, through a user interface of fryer 10. If a dispose cycle is selected by the user, the TPM measurement is cleared. If the user selects not to commence the dispose cycle, the dispose cycle will be initiated after a specific further time of operation, unless a dispose cycle should occur before that specific time.

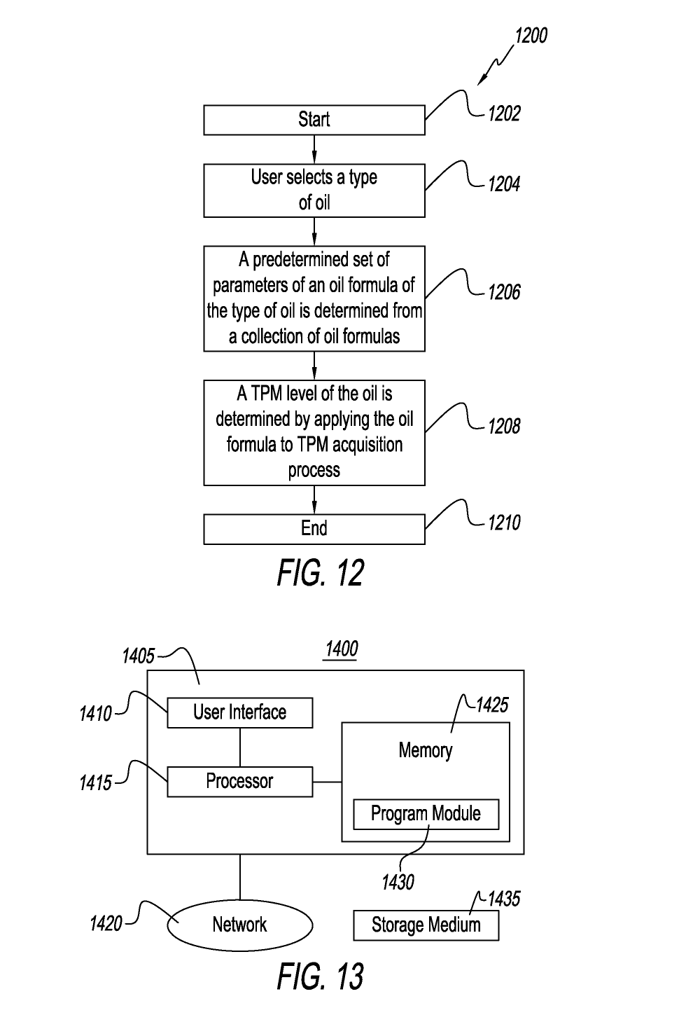

Referring to FIG. 12, a method 1200 of determining a cooking oil formula is shown. In step 1202 method 1200 is commenced. Method 1200 proceeds from step 1202 to step 1204. A user selects a type of cooking oil in step 1204. The user may select the type of cooking oil through a user interface in display panel 31. Method 1200 proceeds from step 1204 to step 1206. A predetermined set of parameters of a cooking oil formula of the type of cooking oil selected in step 1204 is determined from a collection of cooking oil formulas. Method 1200 proceeds from step 1206 to step 1208. A TPM level of the cooking oil selected in step 1204 is determined by applying the cooking oil formula determined in step 1208 to a TPM acquisition process in step 1208. The TPM acquisition process in step 1208 may be step 1126 of method 1100. Method 1200 proceeds from step 1208 to step 1210 to end method 1200.

Various cooking mediums, for example, cooking oil, are available with a variety of chemical formulae. Therefore when measuring TPM level of the liquid form of these cooking mediums, the cooking oil formula and its related dielectric properties must be taken into consideration. The TPM level of cooking oil can be accurately determined by applying the corresponding cooking oil formula to the TPM acquisition process by sensor 400. Each cooking oil formula is represented by a predetermined set of parameters, for example, cooking oil type name. These parameters are used to define a cooking oil type and are referred to as Cooking oil Curve Data. A single or a collection of known Cooking oil Curve Data is transferred to sensor 400 through the serial communication channel from controller 20. This allows sensor 400 to provide TPM levels to an unlimited number of cooking oil formulae. The user is able to simply select the appropriate cooking oil, and its related Cooking oil Curve Data, from the user interface through the serial link. Upon receipt of the command to acquire a TPM measurement, sensor 400 measures TPM in cooking oil contacting sensor 400.

During the TPM measurement activity, the user interface provides a visual display, for example, on display panel 31, that indicates TPM acquisition is in progress. Once the TPM acquisition is complete, the final TPM value is displayed on the user interface for a period of time for fryer pot 15 being filtered and cooking oil quality evaluated. The TPM data is further stored by controller 20 and retrievable for a specific period of time.

Referring to FIG. 13, system 1400 is shown. System 1400 may be controller 20, sensor controller 402 and/or valve controller 600. System 1400 includes a computer 1405 coupled to a network 1420, e.g., the Internet. Computer 1405 includes a user interface 1410, a processor 1415, and a memory 1425. Computer 1405 may be implemented on a general-purpose microcomputer. Although computer 1405 is represented herein as a stand-alone device, it is not limited to such, but instead can be coupled to other devices (not shown) via network 1420.

Processor 1415 is configured with logic circuitry that responds to and executes instructions. Memory 1425 stores data and instructions for controlling the operation of processor 1415. Memory 1425 may be implemented in a random access memory (RAM), a read only memory (ROM), or a combination thereof. One component of memory 1425 is a program module 1430. Program module 1430 contains instructions for controlling processor 1415 to execute the methods described herein, for example, method 1100 and method 1200.

The term “module” is used herein to denote a functional operation that may be embodied either as a stand-alone component or as an integrated configuration of a plurality of sub-ordinate components. Thus, program module 1430 may be implemented as a single module or as a plurality of modules that operate in cooperation with one another. Moreover, although program module 1430 is described herein as being installed in memory 1425, and therefore being implemented in software, it could be implemented in any of hardware (e.g., electronic circuitry), firmware, software, or a combination thereof.

User interface 1410 includes an input device, such as a keyboard or speech recognition subsystem, for enabling a user to communicate information and command selections to processor 1415. User interface 1410 also includes an output device such as a display or a printer. A cursor control such as a mouse, track-ball, or joy stick, allows the user to manipulate a cursor on the display for communicating additional information and command selections to processor 1415. Processor 1415 outputs, to user interface 1410, a result of an execution of the methods described herein. Alternatively, processor 1415 could direct the output to a remote device (not shown) via network 1420.

While program module 1430 is indicated as already loaded in memory 1425, it may be configured on a storage medium 1435 for subsequent loading into memory 1425. Storage medium 1435 can be any conventional storage medium that stores program module 1430 thereon in tangible form. Examples of storage medium 1435 include a floppy disk, a compact disk, a magnetic tape, a read only memory, an optical storage media, universal serial bus (USB) flash drive, a digital versatile disc, or a zip drive. Alternatively, storage medium 1435 can be a random access memory, or other type of electronic storage, located on a remote storage system and coupled to computer 1405 via network 1420.

The method and apparatus for a cooking oil quality sensor of the present disclosure disposes of cooking oil less often leading to a longer cooking oil life span. The longer cooking oil life span leads to cost savings.

It should also be noted that the terms “first”, “second”, “third”, “upper”, “lower”, and the like may be used herein to modify various elements. These modifiers do not imply a spatial, sequential, or hierarchical order to the modified elements unless specifically stated.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. A fryer comprising:
a fryer pot;
a filter pan connected to said fryer pot by a drain conduit and a return conduit forming a filtration loop;
a cooking oil quality sensor being in said filtration loop; and
a controller that controls operation of a filtration cycle of the fryer, said filtration cycle has a circulation sequence and a fill sequence, said circulation sequence circulating cooking oil through said filtration loop and said fill sequence filling said fryer pot with said cooking oil from said filter pan, said controller stops said fill sequence after filling said fryer pot with a predetermined amount of cooking oil during a partial fill and resumes said fill sequence after a predetermined amount time elapses to complete said fill sequence, said cooking oil quality sensor measures a cooking oil quality to obtain a cooking oil quality measurement during said predetermined amount time.

2. The fryer of claim 1, wherein said cooking oil quality sensor is interfaced to said controller in a serial communication channel.

3. The fryer of claim 1, wherein said cooking oil quality is a total polar materials measurement, and wherein said cooking oil quality sensor communicates said total polar materials measurement to said controller.

4. The fryer of claim 1, wherein said controller communicates commands to start and stop said measurement of said cooking oil quality.

5. The fryer of claim 1, wherein said cooking oil quality measurement is a final total polar materials value that is a result of an average of a plurality of samples taken over a second predetermined amount of time during said measurement of said cooking oil quality.

6. The fryer of claim 5, wherein said final total polar materials value is shown on a user interface of the fryer and is stored on said memory.

7. The fryer of claim 1, wherein said filtration cycle is one of a plurality of different types of filtration cycles, wherein said controller has a capability to initiate each of said plurality of different types of filtration cycles, and wherein when one of said plurality of different types of filtration cycles is initiated, a filtration subsystem is notified of a type of said plurality of different types of filtration cycles that was initiated.

8. The fryer of claim 1, wherein said cooking oil quality sensor is notified by said controller when said filtration cycle is initiated.

9. The fryer of claim 1, wherein said controller receives a user input to bypass said cooking oil quality measurement.

10. The fryer of claim 1, wherein when said circulation sequence ends a drain valve of said fryer pot is closed to allow said filling cycle to begin.

11. The fryer of claim 3, wherein after said partial fill, said controller has a filtration subsystem that disables a pump that pumps said cooking oil into said fryer pot and allows a portion of said cooking oil to remain stagnant in said cooking oil quality sensor for a first period of time, and wherein said cooking oil quality sensor measures said total polar materials measurement over a second period of time during said first period of time.

12. The fryer of claim 1, wherein when said cooking oil quality exceeds a predetermined maximum level, said controller initiates a cooking oil dispose cycle to said cooking oil.

13. The fryer of claim 12, wherein when cooking oil quality exceeds a predetermined maximum level, said controller has an option to cancel said dispose cycle.

14. The fryer of claim 1, wherein said filtration cycle is a plurality of filtration cycles and said cooking oil quality is a plurality of total polar materials measurements each taken during one of said plurality of filtration cycles, and wherein said controller has a memory that stores said plurality of said total polar materials measurements.

15. The fryer of claim 14, wherein said plurality of total polar materials measurements are cleared if a dispose cycle is engaged.

16. The fryer of claim 13, wherein if said dispose cycle is canceled, said controller will initiate said dispose cycle after a specific further time of operation, unless a dispose cycle should occur before that specific time.

17. A fryer comprising:
a fryer pot;
a filter pan connected to said fryer pot by a drain conduit and a return conduit forming a filtration loop;
a cooking oil quality sensor being in said filtration loop; and
a controller that controls operation of a filtration cycle of the fryer, said controller having data stored on a memory of a plurality of different types of cooking oil, said controller communicating said data to said cooking oil quality sensor based on one of said different types of cooking oil present in said filtration loop.

18. The fryer of claim 17, wherein each of said plurality of different types of cooking oil has cooking oil curve data that is a predetermined set of parameters used to define each of said plurality of different types of cooking oil.

19. The fryer of claim 18, wherein said cooking oil curve data is communicated to said cooking oil quality sensor through a serial link upon a user selecting one of said plurality of different types of cooking oil through a user interface.

20. The fryer of claim 19, wherein said controller communicates updates of cooking oil curve data to said cooking oil quality sensor.

21. A method of measuring cooking oil quality in a fryer comprising:
commencing a filter cycle in a fryer;
starting a fill sequence to fill a fryer pot with cooking oil;
stopping said fill sequence for a predetermined amount of time;
acquiring total polar materials data during said predetermined amount of time; and
resuming said fill sequence.

22. The method of claim 21, further comprising comparing said total polar materials data to a predetermined set of parameters of said cooking oil.

23. The method of claim 21, further comprising commencing a dispose cycle to dispose of said cooking oil from said fryer when said total polar materials data exceeds a predetermined limit during said comparing.
